(19) United States
(12) Patent Application Publication
(10) Pub. No.: US 2015/0320383 A1
(43) Pub. Date: Nov. 12, 2015

(54) Methods and Systems for Estimating a Size of an Object in a Subject with Ultrasound

(71) Applicant: University of Washington, Seattle, WA (US)

(72) Inventors: Barbrina Dunmire, Seattle, WA (US); Bryan Cunitz, Seattle, WA (US); Oleg A. Sapozhnikov, Seattle, WA (US); Michael R. Bailey, Seattle, WA (US); Franklin Lee, Seattle, WA (US); Mathew D. Sorensen, Seattle, WA (US); Jonathan D. Harper, Bellevue, WA (US); Yasser Haider, Seattle, WA (US)

(21) Appl. No.: 14/705,689

(22) Filed: May 6, 2015

Related U.S. Application Data
(60) Provisional application No. 61/989,376, filed on May 6, 2014.
(60) Provisional application No. 61/989,386, filed on May 6, 2014.

(51) Int. Cl. A61B 8/08; A61B 8/14; A61B 8/00

(52) U.S. Cl. CPC A61B 8/085; A61B 8/5223; A61B 8/0875; A61B 8/0858; A61B 8/4483; A61B 8/14; A61B 8/5207

(57) ABSTRACT

A system and method for determining, via ultrasound, a size of a concretion in a subject are provided. One or more ultrasound pulses are transmitted into a tissue in the subject, which are then reflected from the tissue and received by the ultrasound transducer. A shadow region obscured by the concretion that does not provide reflected signals is generated, and the width of the shadow region is measured. The width of the object is determined based on the width of the shadow region.

### CROSS-REFERENCE TO RELATED APPLICATIONS

This application claims priority to U.S. Provisional Patent Application Ser. No. 61/989,376 filed on May 6, 2014, and to U.S. Provisional Patent Application Ser. No. 61/989,386 filed on May 6, 2014, both of which are hereby incorporated by reference in their entirety.

### GOVERNMENT RIGHTS

This invention was made with government support under Grant Nos. SMST003402 and NIHNIDDK P01, awarded by the National Space Biomedical Research Institute (NSBRI), and DK043881, awarded by the National Institutes of Health. The government has certain rights in the invention.

### BACKGROUND

Size is an important factor in the clinical management of objects in a subject, such as nephrolithiasis and urolithiasis (e.g., kidney stones). Kidney stones smaller than about 5 millimeters (mm) have high spontaneous passage rates and are often observed, whereas kidney stones larger than about 5 mm are less likely to pass spontaneously and thus it is often recommended that patients consider treatment with elective surgery. Accurately sizing kidney stones is thus important to provide an appropriate method of treatment for a subject: underestimation of stone size may result in observation of a kidney stone that is unlikely to pass, and overestimation of stone size may result in surgery to remove the kidney stone that would have passed without intervention. Accurately sizing kidney stones is also important for monitoring stone growth over time, a factor used to determine whether surgery is recommended.

Computerized tomography (CT) is the standard modality used to provide imaging of a kidney stone in a subject. However, CT exposes a subject to ionizing radiation, which is associated with various health effects. The Federal Drug Administration has recently called for a reduction of CT exposure due to such health effects.

Ultrasound is another imaging modality that can be used to image a kidney stone in a patient, and does not pose a risk of radiation exposure. Additionally, ultrasound is inexpensive relative to CT, portable, and widely available. However, ultrasound is currently limited due to factors such as consistent overestimation of stone size relative to CT, low sensitivity and specificity, and user dependence that requires special skills to acquire good quality images. Ultrasound overestimates stone size on average by about 2 mm. Current ultrasound misclassifies up to about 60% of stones smaller than 5 mm as being larger than 5 mm (the size often requiring intervention). Additionally, an incorrect interpretation of multiple stones as one stone may be made using ultrasound.

An ability to more precisely estimate stone size using ultrasound may result in greater adoption of ultrasound for the management of kidney stones. Precise determination of stone size during a patient evaluation is beneficial for clinical decision-making and patient counseling.

### SUMMARY

In accordance with the present invention, a system and a method are defined for determining a size of an object in a body of a subject.

In one embodiment, the method may comprise transmitting an ultrasound pulse to a tissue of the body, receiving signals reflected or scattered from the tissue, generating a shadow region obscured by a reflecting or absorptive object that does not provide reflected signals, measuring a width of the shadow region, and determining a width of the object based on the width of the shadow region.

In some example embodiments, the object may be a kidney stone, a gall stone, a calcification, or an ossification, and the tissue may be a kidney tissue, a urinary tract tissue, a fatty tissue, a bone, or a cyst.

In some cases (e.g., for fluid filled cysts) instead of a bright reflective object with a shadow appearing in an ultrasound image, the object is dark, non-reflecting and non-attenuating with a bright tail behind it caused by the lack of attenuation in the cyst. In this example embodiment, a measurement is made across the bright region. The object shadow may be accentuated, and then the measurement of the shadow may be taken.

In one embodiment, measuring the width of the shadow region comprises determining an axis of propagation of an acoustic beam incident on the object and measuring the width of the shadow region normal to the acoustic beam.

The method may further comprise applying reverse compression to do one or more of the following: enhance brightness of the object and darkness of shadow region, reduce dynamic range, and create a more contrasted image.

In one embodiment, the method uses different angles but does not spatially compound. Measurements of shadow may be made with each angle and repeated frames of the same angle may be averaged. But the frames are not averaged across angles to avoid blurring the shadow.

The shadow may be used in combination with the best measurements taken directly of stone width. For example, the measurements for the shadow and the stone may be averaged, or the smaller of the two used. Both measurements can be provided in the same image and, in certain situations, one may be more accurate than the other. In this vein, if a stone is seen without a shadow, the system may interpret that information as a stone smaller than 5 mm.

In another embodiment, a method to diagnose, prognose, or monitor a kidney stone in a subject is provided. The method may comprise transmitting an ultrasound pulse to a tissue of the body; receiving signals reflected or scattered from the tissue; generating a shadow region obscured by a reflecting or absorptive object that does not provide reflected signals; measuring a width of the shadow region; determining a width of the object based on the width of the shadow region; and diagnosing, prognosing, or monitoring the kidney stone in the subject based on the size of the object.

In another embodiment, a system for measuring an object within a body of a subject is provided. The system comprises an ultrasound transducer and a physical computer readable storage medium. The ultrasound transducer is used to acquire images of the living tissue. The physical computer readable storage medium comprises instructions executable to perform functions to transmit an ultrasound pulse to a tissue of the body, receive signals reflected or scattered from the tissue, generate a shadow region obscured by a reflecting or absorptive object that does not provide reflected signals, measure a width of the shadow region, and determine a width of the object based on the width of the shadow region.

The methods and system may be used to classify a kidney stone as passable or as requiring surgical removal.

The system and method may be used to diagnose, provide a prognosis, monitor, and guide treatment decisions for a kidney stone in a subject.

The system and method may be used for a subject having a concretion within a tissue, including but not limited to nephrolithiasis or urolithiasis. The nephrolithiasis may include any type of kidney stone. The urolithiasis may include any type of urinary stone, within the kidney tissue or the urinary tract. The system and method may be used to determine whether a subject is likely to require surgery to remove the kidney stone, monitor the kidney stone, and make a treatment decision based on a prognosis related to use of the system and method.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a schematic of an exemplary system in accordance with at least one embodiment;

FIG. 2 depicts a simplified flow diagram of an example method that may be carried out to determine a size of an object in a body of a subject, in accordance with at least one embodiment;

FIG. 3 depicts an ultrasound image of a stone including an outline of the stone, in accordance with at least one embodiment;

FIGS. 4a-4c depict ultrasound images of stones and measurements of the stone widths, in accordance with at least one embodiment;

FIG. 5 depicts a table displaying average difference in measured and true stone size as a function of depth, in accordance with at least one embodiment;

FIG. 6 depicts an example ultrasound image including arrows pointing to the stone width and arrows pointing to the shadow width, in accordance with at least one embodiment;

FIG. 7a depicts a graph illustrating measured to true stone width plotted over stone depth in a tissue, in accordance with at least one embodiment;

FIG. 7b depicts a graph illustrating measured to true shadow width plotted over stone depth in a tissue, for three modalities, ray-line imaging (RL), flash angle imaging (SC), and harmonic imaging (HI), in accordance with at least one embodiment;

FIG. 8a depicts a graph illustrating measured stone width to true stone width plotted over stone depth in a tissue for three modalities, ray-line imaging (RL), flash angle imaging (SC), and harmonic imaging (HI), in accordance with at least one embodiment; and

FIG. 8b depicts a graph illustrating measured shadow width to true stone width plotted over stone depth in a tissue for three modalities, ray-line imaging (RL), flash angle imaging (SC), and harmonic imaging (HI), in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

For the present application, the term “stone” may mean any piece of calculus material that may be found in an organ, duct, or vessel of a subject, including stones, stone fragments, and stone dust that may result from the application of shock waves or other therapeutic procedures.

Stones typically appear on ultrasound as a hyperechoic object with a posterior hypeoechoic shadow. The acoustic shadow is a negative return, or a lack of signal, in an otherwise normal image created by the ultrasound pulse echo. As discussed herein, a width of the acoustic shadow may be applied as an alternative measure of stone size. The acoustic shadow may serve as a more accurate indicator of stone size because the edges are not distorted, as can occur with the hyperechoic stone.

### 1. Overview

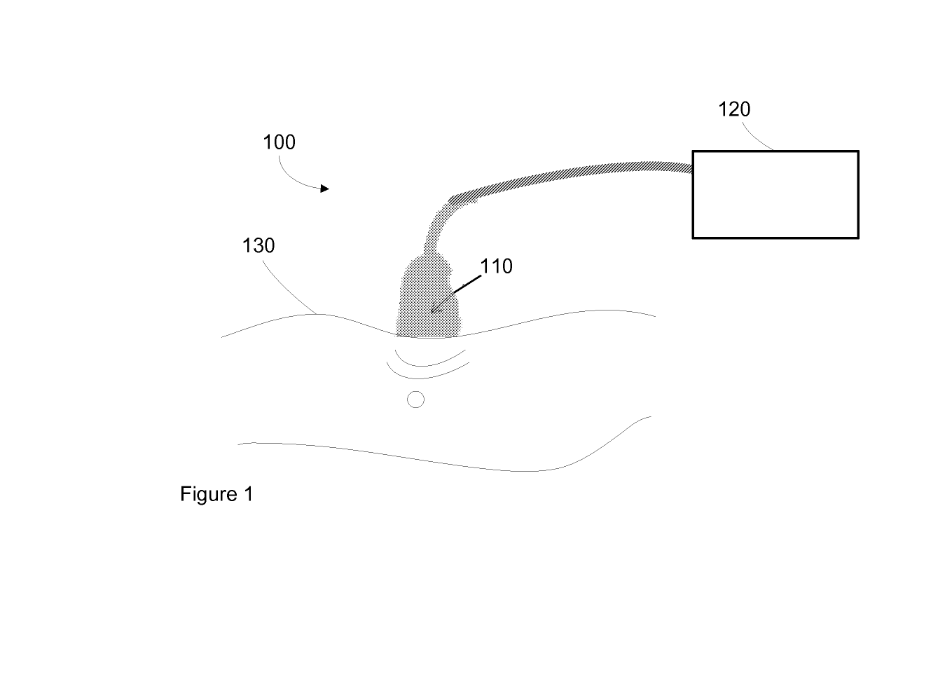

FIG. 1 depicts a schematic of an exemplary system 100 in accordance with at least one embodiment. The system 100 may be used, among other things, to measure an object within a body of a subject. Thus, the system 100 may be used on a subject in vivo. As referenced herein, a subject may be a human subject.

In FIG. 1, an ultrasound system is shown as system 100. The system 100 may include a transducer 110 and a computing system 120. A sample 130 to be imaged is also shown in FIG. 1.

The computing system 120 may include a processor, data storage, and logic. These elements may be coupled by a system or bus or other mechanism. The processor may include one or more general-purpose processors and/or dedicated processors, and may be configured to perform an analysis on the output from the ultrasound system. An output interface may be configured to transmit output from the computing system to a display.

Raw ultrasound data may be analyzed using ray line imaging, flash angle imaging, and harmonic imaging, for example. Ray line or B-mode imaging uses individual elements to direct the acoustic energy to a focus. Resolution is enhanced at the user-selectable focus, at the sacrifice of pre- and post-focal resolution. Placing the focus just proximal to the kidney stone gives the sharpest boundaries between the kidney stone and surrounding medium. Flash angle, or plane wave, imaging averages ultrasound signals captured over multiple angles. The drawbacks with flash angle imaging are that shadowing can be reduced and the stone and shadow boundaries defocused. Because there is no focus, the resolution and signal-to-noise in the region and around the stone is not enhanced. Harmonic imaging is an ultrasound technique that utilizes nonlinear acoustic propagation to improve ultrasound resolution: propagation of finite amplitude sounds results in a distortion of a wave shape and generation of harmonics of the center frequency. The reflected image received by the imager contains these harmonics and harmonic imaging uses the harmonic frequencies which are higher to generate the image, which because of the higher frequency can have higher resolution. The lateral resolution can be improved, but can sacrifice signal-to-noise and penetration depth. All three techniques are generally available on current ultrasound systems, with minor proprietary variations in how they are implemented.

The ray-lines of a B-mode or harmonic scan may be performed with the focus immediately proximal to the stone such that the ray lines overlap to obtain sub-beam width resolution. Alternatively, flash imaging may be used to rapidly obtain frames and avoid tissue movement between frames, and then take an average or a sum of frames from the same angle to accentuate the stone shadow.

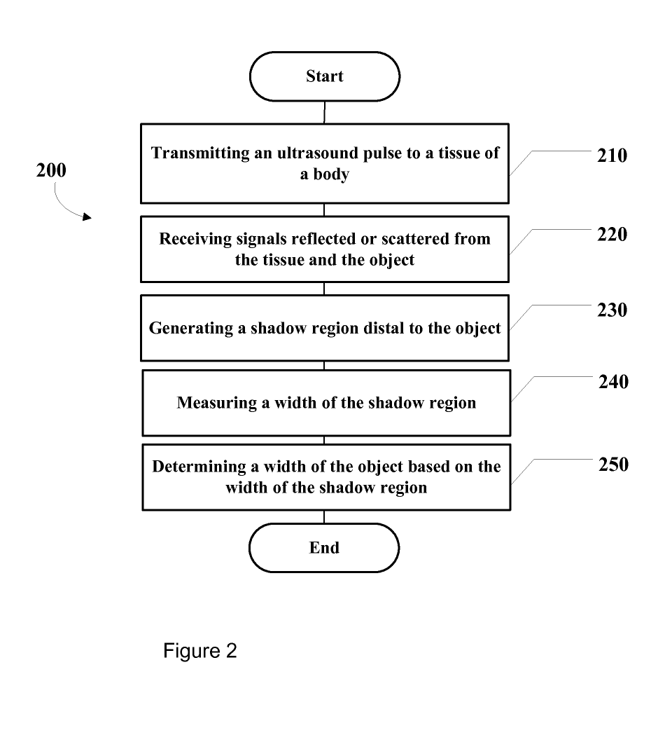

FIG. 2 depicts a simplified flow diagram of an example method that may be carried out to determine a size of an object in a body of a subject, in accordance with at least one embodiment. Method 200 shown in FIG. 2 presents an embodiment of a method that, for example, could be used with the system 100.

In addition, for the method 200 and other processes and methods disclosed herein, the flowchart shows functionality and operation of one possible implementation of the present embodiments. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include a physical and/or non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. Alternatively, program code, instructions, and/or data structures may be transmitted via a communications network via a propagated signal on a propagation medium (e.g., electromagnetic wave(s), sound wave(s), etc.).

The method 200 allows for imaging and determining a size of an object, such as a concretion or kidney stone, using ultrasound. An ultrasound system may be the same or similar to the system 100 of FIG. 1. The method 200 may be used to diagnose, prognose, or monitor a kidney stone in a subject.

Initially, the method 200 includes transmitting an ultrasound pulse to a tissue of the body, at block 210. In operation, a subject is positioned at a designated location to allow for observation of desired biological tissues and concretion of the sample 130. The sample 130 may be observed in vivo, as shown in the example depicted in FIG. 1.

A transducer probe, such as the transducer 110 of FIG. 1, delivers one or more ultrasound pulses into the body. An ultrasound pulse is generally high-frequency (about 1 to 5 megahertz), and travels through one or more tissues in the body. In one example embodiment, the transducer is positioned on the body to deliver an ultrasound pulse through tissues of a kidney. However, the transducer may be positioned on the body to deliver one or more pulses through different tissues, such as the liver or gallbladder, for example.

Transmitting the ultrasound pulse via a transducer may comprise transmitting the plurality of ultrasound pulses to a focus proximal the object, transmitting a plurality of ultrasound pulses at a plurality of angles, or using nonlinear acoustics to receive a reflection at a higher frequency than transmitted. The ultrasound pulse may be focused immediately proximal to the object to measure the width of the shadow region when using ray lines to form the image; alternatively, the ultrasound pulse may not be focused at all when using plane wave imaging, or the ultrasound pulse may be focused far distal if using harmonic imaging and broader ray lines. The ray lines may overlap such that their paths differ by less than the width of the object.

The method 200 then includes receiving signals reflected or scattered from the tissue, at block 220.

The ultrasound pulses travel as waves and hit a boundary between tissues, at which point some of the waves are reflected back to the transducer, while some travel further on until they reach another boundary and are reflected. Additionally, tissue and small objects may scatter diffusely. Signals from the reflected or scattered waves may be received by the transducer and may be relayed to the computing device, such as the computing device 120.

The method 200 includes generating a shadow region obscured by a reflecting or absorptive object that does not provide reflected signals, at block 230.

If a concretion is present in the tissue, the ultrasound pulses will not transmit through the concretion, instead being absorbed by or reflected from the concretion surface. The concretion presence will appear in an image generated from the reflected signals. Because the pulses do not transmit through the concretion, a shadow region behind the concretion will also be generated for an ultrasound image because the shadow region does not provide reflected signals. Distal to a non-reflecting or non-absorbing object a bright region may appear.

Reverse compression may be applied to enhance brightness of the concretion and darkness of the shadow region, reduce dynamic range, and create a more contrasted image. Spatial compounding may be applied to average across frames.

A stone width determination and a determined shadow width may be averaged to obtain a measurement for the stone size, in an example embodiment. In another example embodiment, the smaller value of the two may be applied as the measurement for the stone size.

The method 200 includes measuring a width of the shadow region, at block 240.

The width of the shadow region may be measured, and the width of the concretion may be determined based on the width of the shadow region. The shadow region has a collimated region and a diverging region. The distance from the concretion at which the wave diverges is related to the Rayleigh distance, which is the cross sectional area of the object over the wavelength of the incident acoustic wave. The measurement can be repeated at different frequencies, and narrower band pulses to obtain repetitive approximations of the concretion cross-section.

The method then includes determining a width of the object based on the width of the shadow region, at block 250.

Different analysis techniques, such as B-mode or ray line, flash, and harmonic imaging, and different incident angles between the transducer and the body, may be used to obtain a volume estimate of the size of the concretion and to recreate the concretion shape and dimensions.

The method 200 may further comprise steps to determine an axis of an incident wave, such as identifying and approximating a shape of the object, tracing margins of the shadow region once the shadow region is identified, selecting a location of a width measurement for the shadow region distal the object, and measuring the width normal the beam axis. The width of the shadow region distal the object may then be calculated.

The method 200 may be used to classify a kidney stone as passable or requiring surgical removal. A computing system, such as the computing system 120, may execute instructions to plot the results on a display.

### 2. Example Embodiments

An evaluation was performed to investigate the use of the stone acoustic shadow to reduce overestimation of stone size.

Ten human calcium oxalate monohydrate stones (“kidney stones”) ranging in size from 3 to 12 mm were used for the study. All of the kidney stones were rehydrated for at least 48 hours before the stone size measurements were captured. The kidney stones were placed on an attenuative gel phantom to reduce scatter from an applied ultrasound, and were then immersed and imaged through a water bath. A transducer was mounted and oriented such that the maximum measured width of the stone was aligned with the long axis of the ultrasound probe. Image guidance was used to optimize the probe alignment with the stone and to verify stone depth. The theoretical depth was estimated to be within 2 mm from the true depth. The ultrasound instrument utilized preprogrammed settings for abdominal imaging with spatial compounding turned off.

The stones were imaged at three depths from the transducer: 6 centimeters (cm), 8 cm, and 10 cm. B-mode images of the stones were captured using a commercial ultrasound instrument and a programmable instrument based on the Verasonics data acquisition system. The collected images were then loaded into MATLAB where the left and right edges of the stone were marked with calipers.

Images captured included both moderate and high gain settings. For purposes of the study, high gain was defined as about 80% peak saturation of the stone and moderate gain was defined as about 65% peak saturation of the stone. The settings were consistent for all ten stones, and the stone position was not moved between acquisitions from the images.

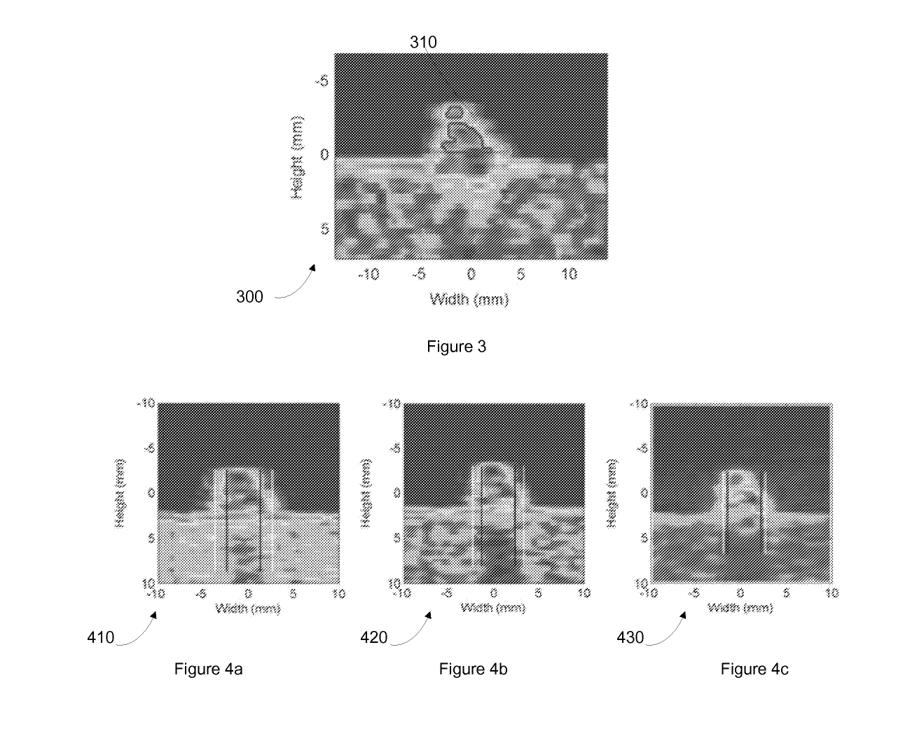

Each B-mode image was loaded into MATLAB and the user manually identified the approximate center of the stone. The program then interrogated a 15 mm×15 mm region surrounding the central coordinate using a pre-set threshold value based on the ultrasound gray scale signal intensity (ranging from 0 to 255). Above the threshold, the pixel was identified and assigned as a stone. The contour program in MATLAB was then used to outline the stone, as depicted in FIG. 3. FIG. 3 depicts an ultrasound image 300 of a stone including an outline 310 of the stone. The size of the stone was calculated as the distance between the left and right edge coordinates. The threshold was adjusted from an intensity value of 30 to 180, in intervals of 5, to determine the intensity value that returned the least error in stone size for each stone individually. The average threshold result for each group of 10 stones was used to calculate an average error and standard deviation.

Overestimation for manual measurement of stone size is depicted in FIGS. 4a-4c. In FIGS. 4a-4c, white lines represent the manual measurement and black lines represent the true stone size. As shown in the image 410 of FIG. 4a, stone size measured manually was overestimated an average of 1.9±0.8 mm (commercial ultrasound at moderate gain), in the image 420 of FIG. 4b, stone size measured manually was overestimated an average of 2.1±0.9 mm (commercial ultrasound at high gain), and in the image 430 of FIG. 4c, stone size measured manually was overestimated an average of 1.5±1.0 mm (research based ultrasound at moderate gain).

With the commercial system, overestimation increased with increasing depth (p=0.02). At moderate gain, stone size measurement increased by 23% from 6 to 8 cm and 27% from 8 to 10 cm. The results at high gain demonstrated a similar trend (p=0.02), as stone size measurement increased by 22% from 6 to 8 cam and 19% from 8 to 10 cm.

With the research based ultrasound machine, the stone size measurement did not significantly change as a function of depth (p=0.99). Stone size measurement increased by 18% from 6 to 8 cm and decreased by 15% from 8 to 10 cm.

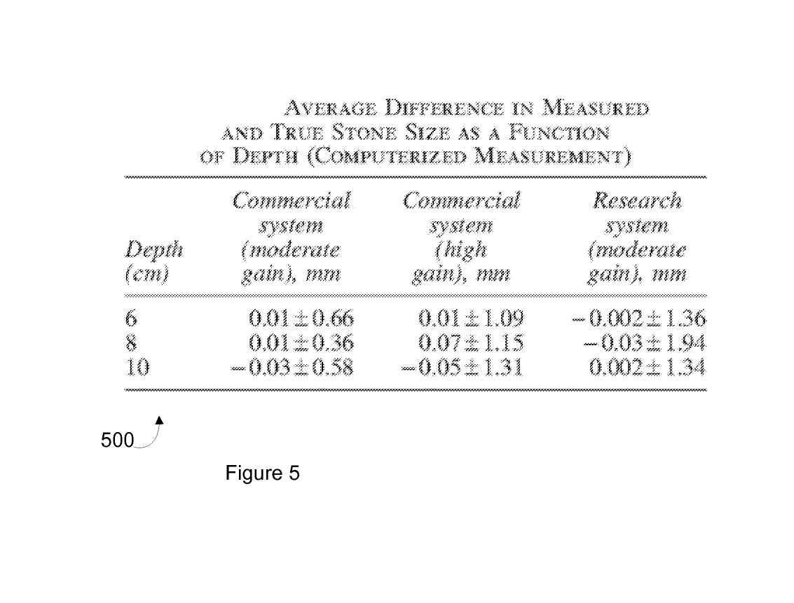

Stone measurement as a function of gain was examined within the commercial system, depicted in FIG. 5. FIG. 5 depicts a table 500 displaying average difference in measured and true stone size as a function of depth. Increasing the gain at a given depth tended to result in greater overestimation of stone size, but was not statistically significant (p=0.6). Stone size overestimation increased by 18% from the moderate to high gain setting at both 6 and 8 cm depths. Overestimation increased by 9% from the moderate to high gain setting at 10 cm depth. Average discrepancy between the computer calculated stone size and true stone size was minimized to −0.01±0.5 mm (commercial ultrasound at moderate gain), 0.01±1.2 mm (commercial ultrasound at high gain), and −0.01±1.5 mm (research based ultrasound). These threshold settings were not consistent across stone, depth, system, or gain setting, however.

Based on the results of the evaluation illustrated in FIG. 5, it may be preferable to use a low to moderate gain setting for improved accuracy of sizing concretions such as kidney stones. Increasing gain may artificially expand the stone border and lead to overestimation.

In another evaluation, forty-five human calcium oxalate monohydrate stones were provided, ranging in size from 1-10 mm, with an equal number of stones (five) per millimeter. Photographs were taken of the stones, including a millimeter ruler for reference. The images were uploaded into MATLAB for determining the true stone size.

Each stone was then placed on top of an agar-based tissue-mimicking phantom in a water bath, and ultrasound images were captured at three transducer-to-stone depths: 6 cm, 10 cm, and 14 cm. The transducer was mounted downward and oriented such that the maximum measured width of the stone was aligned with the long axis of the probe. From the digital images, stone and shadow measurements were made by four reviewers blinded to true stone size.

The ultrasound images were captured with a Verasonics data acquisition system using a 128 element C5-2 curve linear imaging probe operating at 3.2 MHz. Three different imaging techniques were used for comparison of stone sizing accuracy: B-mode imaging, spatial compound imaging, and harmonic imaging. Thus, at each depth, images were captured from all three modalities.

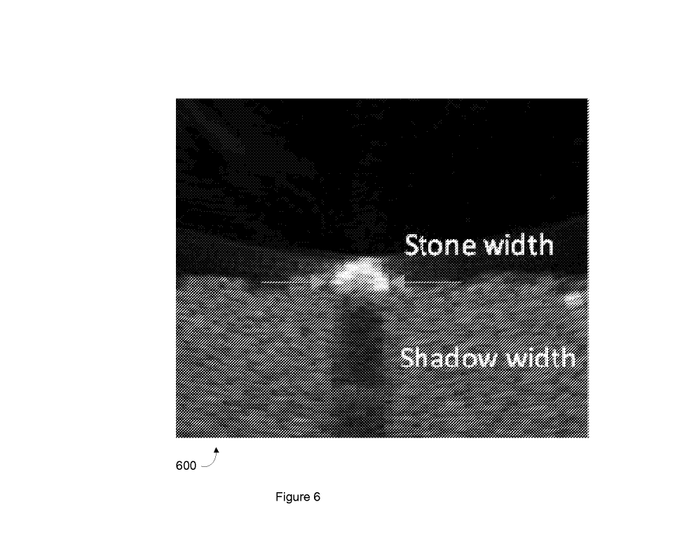

The stone width was measured as the greatest linear distance between two hyperechoic edges. The width of the posterior acoustic shadow (shadow width) was measured as the distance between two hyperechoic edges. FIG. 6 depicts an example ultrasound image 600 including arrows pointing to the stone width and arrows pointing to the shadow width. Because the acoustic shadow spreads laterally the further away it is from the stone, the shadow edges were measured close to the stone, typically less than 1 cm from the stone. Measurements were made by four reviewers, all blinded to the true stone size. The forty-five stones, four reviewers, three depths, and three imaging modalities provided a total of 2025 cases.

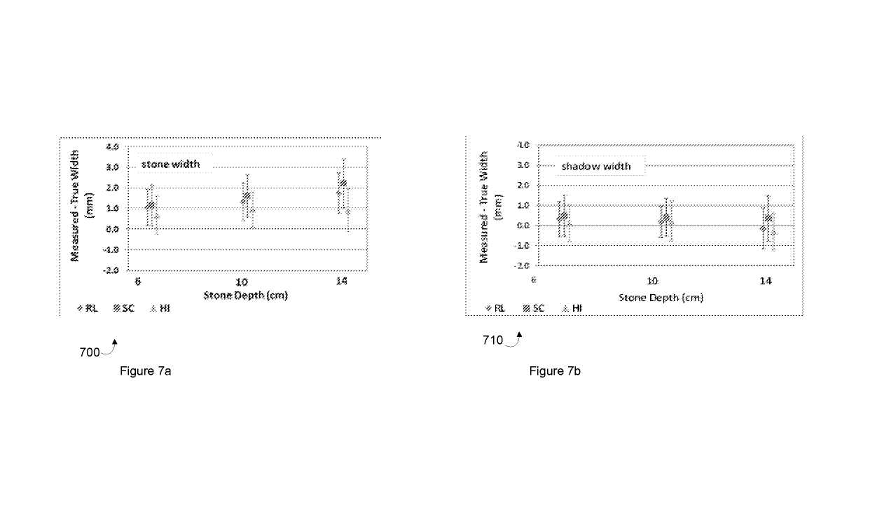

The stone width results for the three imaging modalities, averaged over all stones, are shown in FIG. 7a. FIG. 7a depicts a graph 700 illustrating measured to true stone width plotted over stone depth in a tissue. Stone width measurements overestimated stone size in all cases. Harmonic imaging was more accurate than B-mode ray line imaging in determining true stone size, but this did not reach statistical significance.

The average error in stone size using the shadow width measurement was less than 0.5 mm for all depths and modalities, as shown in FIG. 7b. FIG. 7b depicts a graph 710 illustrating measured to true shadow width plotted over stone depth in a tissue, for three modalities, ray-line imaging (RL), SC imaging (SC), and harmonic imaging (HI). Measuring the shadow was more accurate than measuring the stone width for all three modalities. Moreover, shadow measurement did not worsen with depth, and thus had improved accuracy compared to measuring the stone width as depth increased.

When measuring the stone width, three stones (15%) at 6 cm and up to 10 stones (50%) at 14 cm were misclassified as greater than 5 mm when true stone size was equal to or smaller than 5 mm. The average size overestimation for the misclassified stones was 2.2±0.9 mm. Only one stone (5%) was misclassified as smaller than 5 mm when true stone size was greater than 5 mm. A measurement was not reported for 3% (53 of 2025) of the cases, all of which were under 5 mm. Additionally, a shadow was not consistently present for small stones. A measurement was not reported for 24% (385 of 2025) of the cases, all of which were under 5 mm.

When measuring the shadow width, one stone (5%) up to three stones (15%) were overclassified as greater than 5 mm when true stone size was equal to or less than 5 mm. The average size overestimation for the misclassified stones was 0.8 ±0.2 mm. A maximum of three stones (15%) were under-classified as less than 5 mm when the true stone size was greater than 5 mm.

The results showed that B-mode ray line imaging shadow width and harmonic imaging stone width were the most accurate and reliable methods for sizing kidney stones over all depths. Two stones (6 cm depth and 14 cm depth) and one stone (10 cm depth) out of 19 stones less than 5 mm were misclassified as greater than 5 mm. Up to three stones greater than 5 mm were misclassified as less than 5 mm.

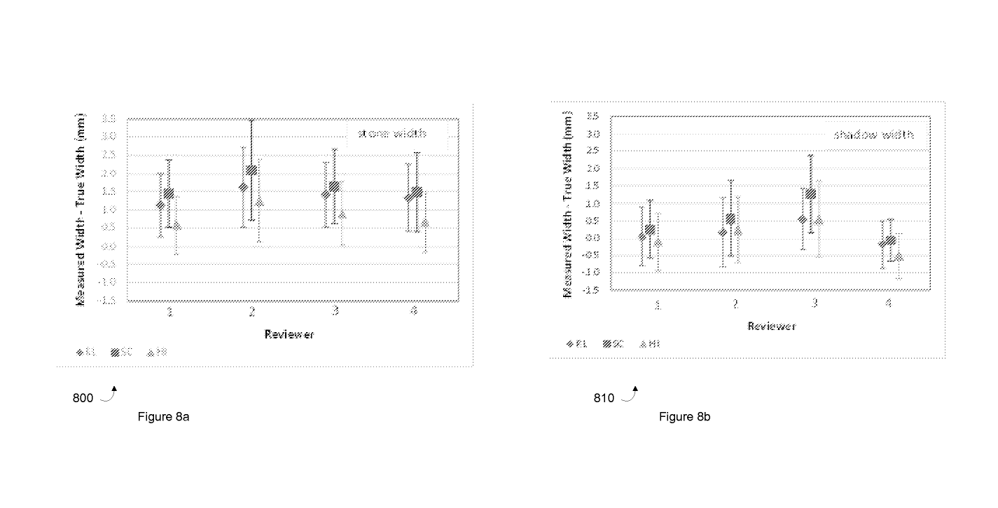

Stone measurement error by each reviewer across imaging modality is depicted in FIGS. 8a-8b. FIG. 8a depicts a graph 800 illustrating measured stone width to true stone width plotted over stone depth in a tissue for three modalities, ray-line imaging (RL), SC imaging (SC), and harmonic imaging (HI). FIG. 8b depicts a graph 810 illustrating measured shadow width to true stone width plotted over stone depth in a tissue for three modalities, ray-line imaging (RL), SC imaging (SC), and harmonic imaging (HI). The intra-class correlation analysis was over 0.80 for all cases except the harmonic imaging shadow at 14 cm, which had reduced signal, as discussed in the methods. For all four reviewers, the most accurate and precise measurements were taken by measuring the stone shadow. When the stone was measured directly, harmonic imaging was the most accurate and flash rate was the least accurate.

These results show that measuring the width of the stone using ultrasound overestimates true stone sizes, and the extent of overestimation increases with depth. The average overestimation was about 1.5-2.0 mm. Stone size accuracy was significantly improved by measuring the acoustic shadow width. Using this technique, all three imaging methods were similar in accuracy. Stone size accuracy was further improved by taking the smallest of ray line shadow width versus harmonic imaging stone width, the two most accurate and reliable methods. Across all depths, stone size accuracy based on this combination neared zero error and the ±1 mm precision of clinical CT imaging. The presence of a stone shadow did diminish with decreasing stone size and increasing depth. The lack of a stone shadow in combination with a ray line stone measurement <5 mm, however, was also a reliable indicator of a stone less than 4 mm.

In addition to improving stone size accuracy, the use of the shadow reduced the misclassification of stones as greater or less than 5 mm. The results were consistent for all four reviewers who performed the measurements. Conventional B-mode gave the highest correlation between the users. It is expected that the findings will be consistent in vivo. Initial preliminary results from four subjects in an ongoing prospective study show that, between the four subjects, there were 13 kidney stones, 85% of which produced an acoustic shadow. Stone width resulted in an average overestimation of 1.3±1.1 mm, while shadow width resulted in an overestimation of 0.4±0.6 mm.

As discussed above, the measurement of the stone shadow may be used to diagnose, provide a prognosis, monitor and guide treatment decisions for an object, such as a kidney or gall stone, in a body of a subject. The treatment may include medical monitoring or surgical intervention.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

What is claimed is:

1. A method for determining a size of an object in a body of a subject comprising:
transmitting an ultrasound pulse to a tissue of the body;
receiving signals reflected or scattered from the tissue and the object;
generating a shadow region distal to the object;
measuring a width of the shadow region; and
determining a width of the object based on the width of the shadow region.

2. The method of claim 1, further comprising:
applying reverse compression to do one or more of the following: enhance brightness of the object and darkness of shadow region; reduce dynamic range; create a more contrasted image; and remove spatial compounding to average across frames.

3. The method of claim 1, wherein transmitting the ultrasound pulse comprises transmitting a plurality of ultrasound pulses at a plurality of angles and using individual image frames for determining the width of the object, without averaging the image frames.

4. The method of claim 1, wherein measuring the width of the shadow region comprises determining an axis of propagation of an acoustic beam incident on the object and measuring the width of the shadow region normal to the acoustic beam.

5. The method of claim 4, further comprising:
altering an angle of incidence for transmission of the ultrasound pulse; and
generating a plurality of measurements of the shadow region width.

6. The method of claim 1, further comprising:
measuring one or more distances from the object to a point at which a wave diverges at varying frequencies; and
determining, from the one or more distances, a cross-sectional area of the object over the wavelength of the wave.

7. The method of claim 1, wherein the object is one of a kidney stone, gall stone, a calcification, and an ossification.

8. The method of claim 7, wherein the tissue is one of a kidney tissue, a fatty tissue, a bone, and a cyst.

9. The method of claim 1, further comprising:
determining the size of the object as an average of the width of the shadow region and the measured width of the object by generating harmonics.

10. The method of claim 1, further comprising:
determining an axis of an incident wave;
approximating a shape of the object;
tracing margins of the shadow region;
selecting a location of a width measurement distal the object; and
measuring the width normal the axis.

11. The method of claim 1, wherein the method is used to determine whether a kidney stone is passable or requiring surgical removal.

12. A method to diagnose, prognose, or monitor a kidney stone in a subject, comprising:
transmitting an ultrasound pulse to a tissue of the body;
receiving signals reflected from the tissue;
generating a shadow region obscured by a reflecting or absorptive object that does not provide reflected signals;
measuring a width of the shadow region;
determining the width of the object based on the width of the shadow region; and
diagnosing, prognosing, or monitoring the kidney stone in the subject based on the size of the object.

13. The method of claim 12, further comprising:
applying reverse compression to do one or more of the following: enhance brightness of the object and darkness of shadow region; reduce dynamic range; create a more contrasted image; and remove spatial compounding to average across frames.

14. The method of claim 12, wherein transmitting the ultrasound pulse comprises transmitting a plurality of ultrasound pulses at a plurality of angles and using individual image frames for determining the width of the object, without averaging the image frames.

15. The method of claim 12, wherein the method is used to determine whether the kidney stone is passable or requiring surgical removal.

16. A system for measuring an object within a body of a subject comprising:
an ultrasound transducer; and
a physical computer-readable storage medium;
wherein the physical computer-readable storage medium has stored thereon instructions executable by a device to cause the device to perform functions to determine the size of an object in a body, the functions comprising:
transmitting an ultrasound pulse to a tissue of the body;
receiving signals reflected from the tissue;
generating a shadow region obscured by a reflecting or absorptive object that does not provide reflected signals;
measuring a width of the shadow region; and
determining the width of the object based on the width of the shadow region.

17. The system of claim 16, wherein the transducer transmits a brightness-mode (B-mode) ultrasound pulse proximal to the object such that ray lines overlap to obtain sub-beam width resolution.

18. The system of claim 16, wherein frames generated using the B-mode ultrasound pulse are averaged or summed to accentuate the shadow region.

19. The system of claim 17, the functions further comprising:
applying reverse compression to do one or more of the following: enhance brightness of the object and darkness of shadow region; reduce dynamic range; and create a more contrasted image.
